(19) United States
(12) Patent Application Publication
(10) Pub. No.: US 2003/0100820 A1
(43) Pub. Date: May 29, 2003

(54) Medical system architecture having a component-oriented architecture for diagnostics and documentation

(75) Inventors: Thomas Birkhoelzer, Radolfzell (DE); Frank Krickhahn, Herzogenaurach (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/303,483

(22) Filed: Nov. 25, 2002

(30) Foreign Application Priority Data
Nov. 26, 2001 (DE) 10157633.1

(51) Int. Cl. A61B 5/00; G06F 17/00; G06F 7/00

(52) U.S. Cl. 600/300; 707/104.1

(57) ABSTRACT

A medical system architecture has a workstation for acquiring and/or post-processing data and/or examination images, the workstation including an image processing system based on a component-oriented architecture, into which procedures for producing and manipulating text information (findings) are linked by means of generic objects and interfaces, so that the corresponding procedures can be flexibly replaced without modification at the medical image processing system.

### BACKGROUND OF THE INVENTION

1. Field of the Invention

The present invention is directed to a medical system architecture having a workstation for acquiring and/or post-processing data and/or examination images, the workstation including an image processing system based on a component-oriented architecture.

2. Description of the Prior Art

The book Bildgebende Systeme für die medizinische Diagnostik, edited by H. Morneburg, 3rd Edition, 1995, pages 684 ff. discloses medical system architectures, referred to as PACs (Picture Archival and Communication Systems) wherein image viewing stations and image processing stations, referred to as workstations, are connected to one another via an image communication network for retrieving patient data and images generated by modalities. The images can be diagnosed by experts with this workstation.

The diagnosis of medical images requires the use of tools or applications for generating structured or unstructured information, i.e. essentially text or text elements. Different tools are employed for this purpose dependent on the purpose. The system thus must be able to link in different tools without making use of complicated procedures.

It is well known that the tools for producing the diagnosis be either permanently linked into the system or be completely detached therefrom such as, for example, the radiology information system (RIS). Currently available programs do not offer the flexibility of replacing one tool with another, for example replacing the WinWord text processing system with some other tool for generating structured information.

Such problems arise because different modalities have different scenarios with different levels of inter-activity and automation. The various users do not know what applications or tools they will need in the future. Moreover, a nearly seamless data integration must be assured in the departments (HIS/RIS).

### SUMMARY OF THE INVENTION

An object of the present invention is to provide an image processing system of a medical workstation that flexibly allows for expansions and modifications so that one tool can be easily replaced by another, so that various tools can be linked in without complicated procedures. A reporting framework must be able to manipulate with various applications (tools). It must be independent from the tools that are employed. A “framework” is a term from object-oriented programming. It is a re-employable basic design structure that is composed of abstract and concrete classes and supports the production of applications.

This object is inventively achieved in a system of the type initially described having means for producing and manipulating text information (findings) are linked into the image processing system by means of generic objects and interfaces, so that the corresponding means can be flexibly replaced without modification at the medical image processing system.

“Means for producing and manipulating text information (findings)” are software applications or software components whose applied purpose to generate and manipulate textual information. Examples are Microsoft Word or FrameMaker but such means also can be specific medical applications.

“Generic objects” are objects that can be very flexibly and universally employed. Generic basically means that the object is realized without customization to a specific application. Generic approaches are encountered especially often in information science where it is a matter of flexibility and re-employability are important.

The text information can be represented by a generic object that contains unambiguous features for the identification thereof and about the means to be employed.

It has proven advantageous for the image processing system to identify the means to be employed and to control such means via a means-specific component that is linked to the image processing system.

The tools can be simply linked in and replaced by integration of the respective generic component when the means are represented by a generic component with generic methods, functions and/or interfaces. The tool-specific parts of the program code are maximally encapsulated and have no interactions whatsoever with the system itself.

Inventively, the means for producing and manipulating text information can be text processing systems, and/or tools, and/or applications.

In a medical system architecture having a workstation for acquiring and/or for post-processing data and/or examination images, the object is also inventively achieved by the workstation having an image processing system based on a component-oriented architecture, interchangeable means for producing and processing text information (findings) to which an object that contains identification criteria and references to the means to be employed is allocated, a device (handler dispatcher) for identification of the means to be employed on the basis of the identification criteria, and a control device for the means for executing control on the basis of the references via a means-specific component linked to the image processing system. The link-in of means (tools) via a generic reporting component enables a good integration without specific dependencies, and thus enables maximum interchangeability.

### DESCRIPTION OF THE DRAWINGS

FIG. 1 schematically illustrates an example of a system architecture of a hospital network.

FIG. 2 is a schematic illustration of a part of an inventive medical image processing system of the workstation according to FIG. 1.

FIG. 3 is an example of an object that represents the reports in a databank.

FIG. 4 is an illustration of the data storage with different formats in the databank, with an appertaining reporting application identified by an ID.

FIG. 5 is an example of another object that represents the reports in a databank.

FIG. 6 is an illustration of the data storage with different formats in the databank, with an appertaining reporting application is identified by a UID.

FIG. 7 illustrates a derivation of the handler classes from a basic class.

FIGS. 8-9 illustrate the functioning of a WinWord Report Handler.

FIG. 10 is an illustration of the registration of the report applications at the report dispatcher.

### DESCRIPTION OF THE PREFERRED EMBODIMENTS

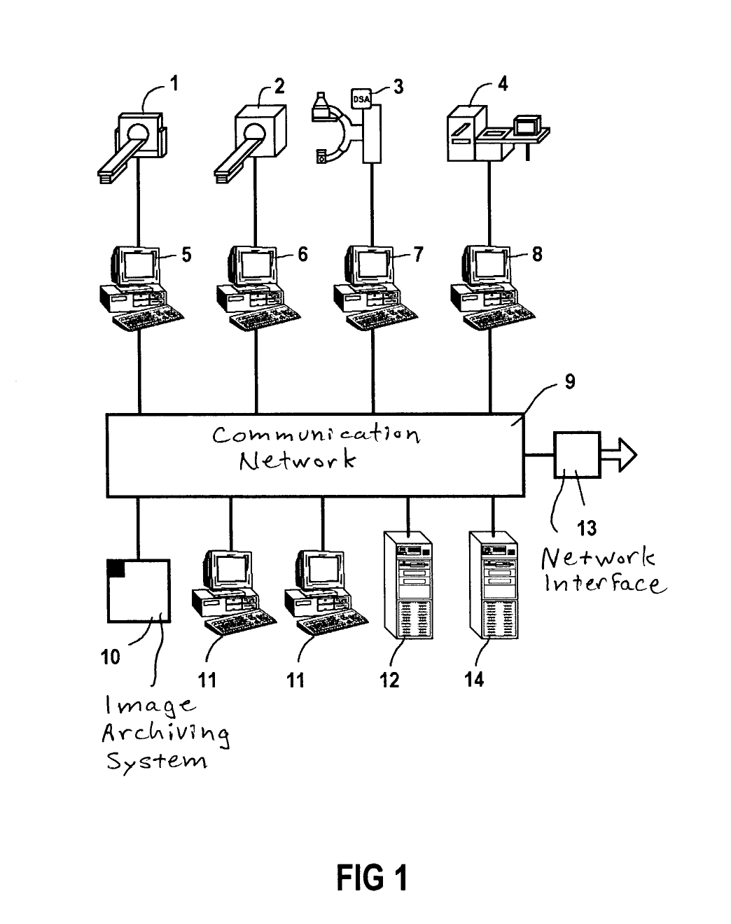

FIG. 1 shows the system architecture of a hospital network as an example. The modalities 1 through 4 serve for the acquisition of medical images and, as image-generating systems, may be, for example, a CT unit 1 for computed tomography, an MR unit 2 for magnetic resonance, a DSA unit 3 for digital subtraction angiography and an X-ray unit 4 for digital radiography. Operating consoles 5 through 8 of the modalities or workstations are connected to these modalities and the acquired medical images can be processed and locally stored therewith. Patient data belonging to the images also can be entered.

The operating consoles 5 through 8 are connected to a communication network LAN/WAN backbone for the distribution of the generated images and for communication. Thus, for example, the images generated in the modalities 1 through 4 and the images further-processed in the operating consoles 5 through 8 can be stored in a central image storage and image archiving systems 10 or can be forwarded to other workstations.

Further viewing workstations 11 that have local image memories are connected to the communication network 9 as diagnosis consoles. Such a viewing workstation 11 is, for example, a very fast small-sized computer employing one or more fast processors. The images that have been acquired and that are stored in the image archiving system 10 can be subsequently retrieved for diagnosis in the viewing workstations 11 and can be deposited in the local image memory thereof, being then directly available therefrom for the diagnostician working at a viewing workstation 11.

Servers 12, for example a patient data server (PDS), a file server, program server and/or EPR server, are also connected to the communication network 9.

The image and data exchange via the communication network 9 ensues according to the DICOM standard, an industrial standard for the transmission of images and further medical information between computers, so that a digital communication is possible between diagnosis and therapy devices of different manufacturers. A network interface 13, via which the internal communication network 9 is connected to a global data network, for example the World Wide Web, can be connected to the communication network 9, so that the standardized data can be exchanged worldwide with different networks.

An RIS server and/or HIS server 14 with which the operating consoles 5 through 8 can communicate by means of the communication network 9 via TCP/In protocols can also be connected to the communication network 9.

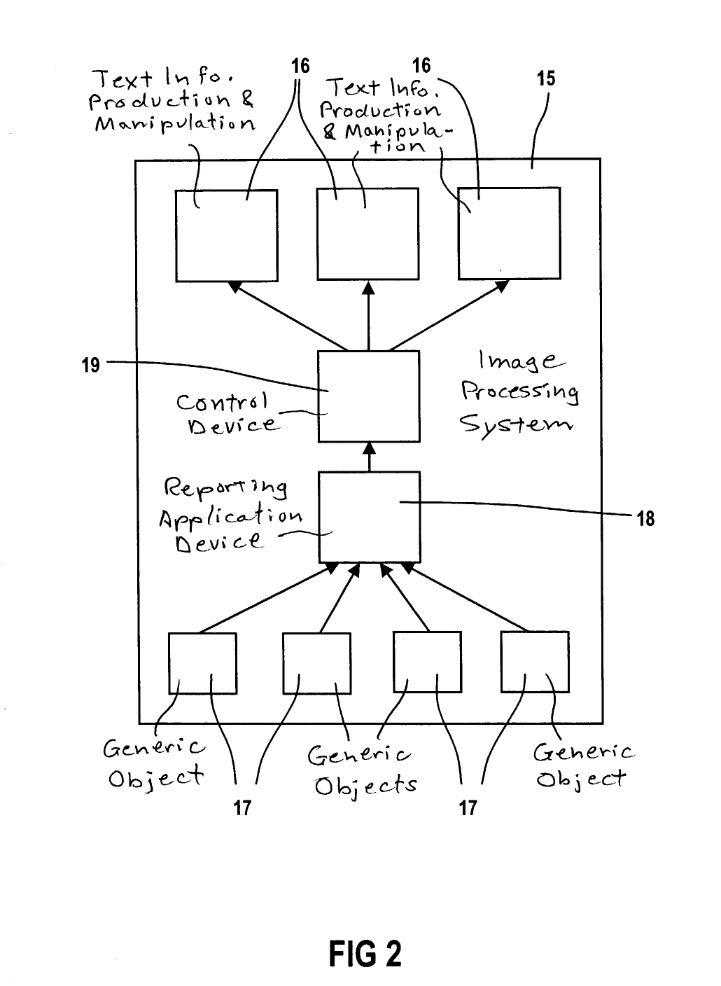

Such workstations (consoles) 5 through 8 and 11 each have an image processing system 15 that is schematically shown in FIG. 2. Such a medical image processing system 15 is based on a component-oriented architecture into which means 16—tools or systems—for producing or manipulating text information (findings), for example text processing systems, are linked by means of generic objects and interfaces, so that the corresponding means 16 can be flexibly replaced without modification of the medical image processing system 15.

The findings are represented by generic objects 17 that contains unambiguous features for their identification and about the means 16 or tool to be employed. Using a device 18—a reporting application—the system identifies the means 16 to be employed and, using a control device 19, controls this means 16 to be employed via a tool-specific component that is linked to the system.

The tool or the means 16 are likewise represented by a generic component with generic methods, functions and interfaces. The employed means 16 can thus be linked in and replaced simply by integration of the respective generic component. The means-specific parts of the program code are maximally encapsulated and have no interactions whatsoever with the system itself.

The linking-in of means 16 via the generic reporting component enables a good integration without specific dependencies and, thus, allows maximum interchangeability.

In this context, generic means that a realization ensues without customization to a specific application. Generic approaches can generally be found especially often in information science when flexibility and re-employability are necessary.

An application as well as a tool in the sense of the present patent application are executable computer programs. An application serves as tool for fulfilling a purpose, just like a tool as well. More complex items such as, for example, multi-layer architectures are more likely to be referred to as system.

These problems are solved by the inventive fashioning of the image processing system.

The storing of reports in the patient object hierarchy as a generic object functions as format-independent place holder for reports in various formats such as, for example, WinWord, PowerPoint and DICOM-SR.

The reports are controlled by means of an expandable, component-based framework.

The generic reporting object can be composed of an envelope. The advantage of employing the DICOM-SR structure is the compatibility with the medical framework structure.

The generic reporting object has a content (application-specific structure) that, for example, can include a “simple” DICOM-SR structure or a pointer.

The generic reporting object can be a mere space holder. It contains an unambiguous identification and a type information. The unambiguous identification is required as a pointer to the real report.

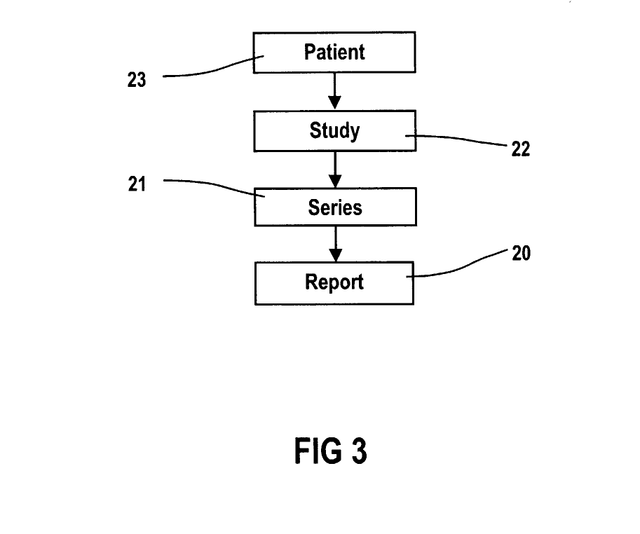

FIG. 3 shows a report 20 within an object tree. A report 20 is the part of a series 21 in the DICOM-SR specification. This series 21 is turn belongs to a study 22 of a patient 23.

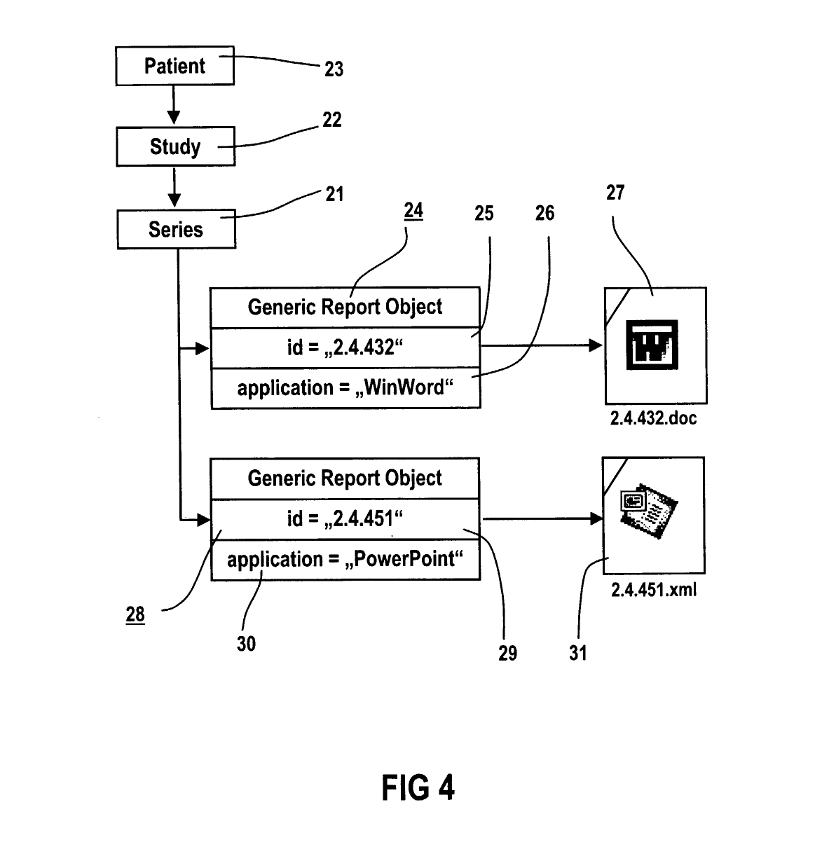

The report 20 is shown in greater detail in FIG. 4. In this example, it is composed of a first generic object 24 to which an identifier (ID) 25 is allocated. The application 26 to be employed, WinWord in this case, so that a first record 27 of the report 20 to which the ID 25 points can be called with the correct application. A second generic FNI object 28 is provided with a send-ID (UID) referenced 30, so that the appertaining, second record 31 of the report 20 is opened with the application to be employed, such as PowerPoint.

The identifiers (IDs) can be numbers, names or other unambiguous features that are employed for recognizing specific devices, users, persons, general procedures or other elements in a computer or a program. The send-ID (UID) is a unique identifier, an unambiguous and one-time identifier. PowerPoint reporting is an application for PPT-based generation of clinical reports.

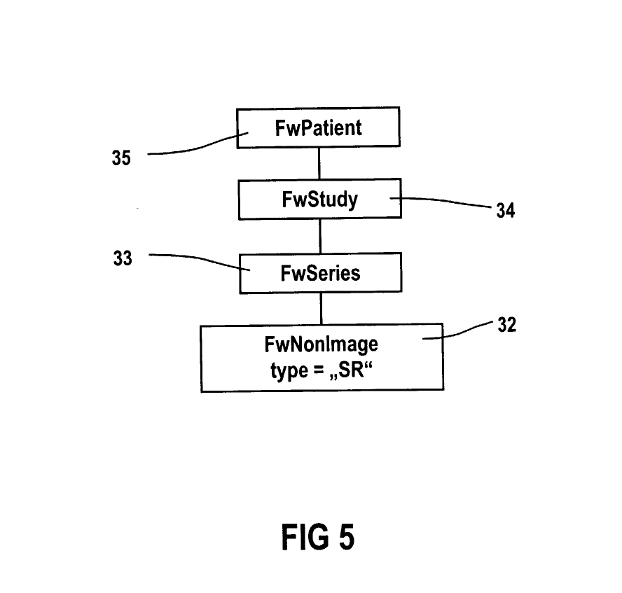

FIG. 5 shows an implemented prototype within an object tree. FwNonImage 32 (FNI), an object of a framework that does not represent an image, is required as a generic reporting object with the database identification as unambiguous identifier. FNI 32 is part of an FwSeries 33. This in turn belongs to an FwStudy 34 of an FwPatient 35.

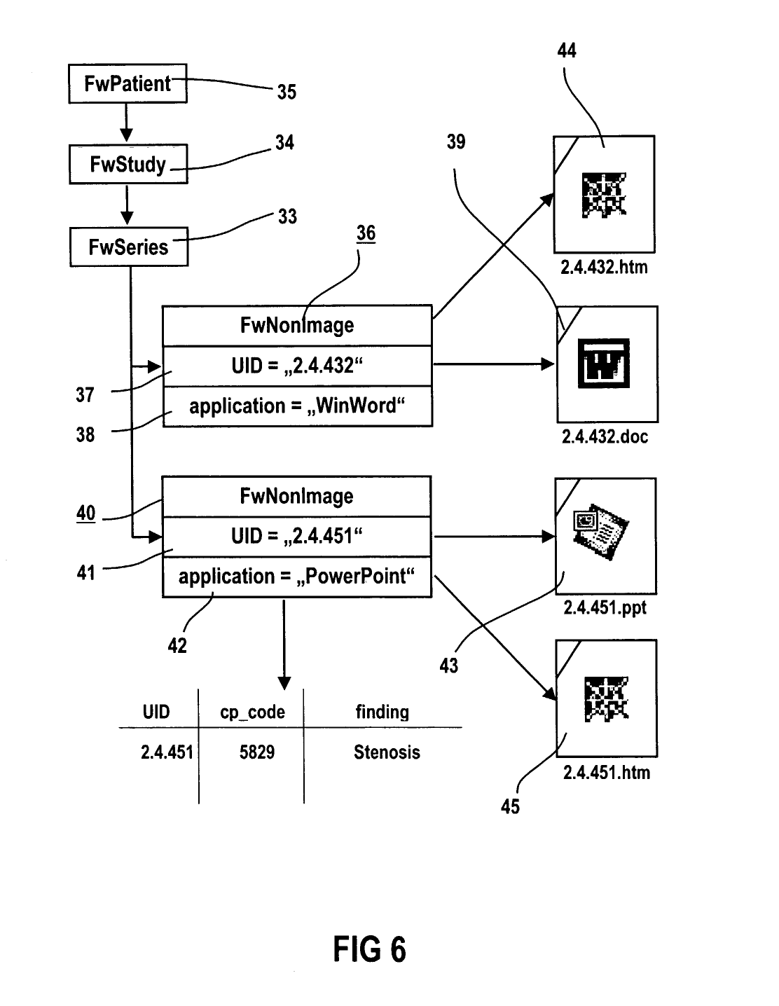

The send-ID (UID) 37 is required as unambiguous identifier, this being allocated to a first generic FNI object 36 shown in FIG. 6. Reference 38 identifies the application to be employed, WinWord in this case, so that the FwReport 39 to which the UID 37 refers can be called with the correct application. A second generic FNI object 40 is provided with a UID 41 and a reference 42, so that the appertaining FwReport 43 is opened with the application to be employed, to wit PowerPoint. So that the user is not made aware at all that the reports are internally handled completely differently according to their properties, HTML representations 44 and 45 can be produced, for example, from each report 39 and 43 by the appertaining reporting application 38 and 42 in order to be able to display the reports in the Internet browser.

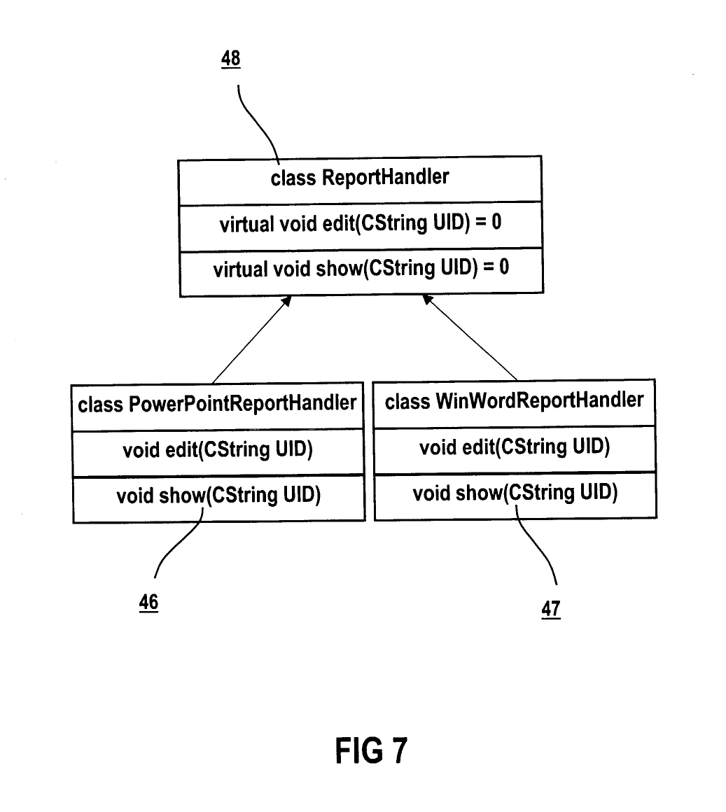

A handler class, as shown in FIG. 7 for PowerPoint (46) and WinWord (47), exists for each implemented application. All handler classes 46 and 47 are derived from a common basic class 48. This common basic class 48 declares [or: explains] all methods that must be implemented by the handler classes 46 and 47. All methods that must act on reports must at least employ the unambiguous identifier of the report to be processed. By identifying the methods in the basic class 48 as virtual, these methods of this object can be overloaded into the handler classes 46 and 47 by a same-name method of a derived object.

A handler is also referred to as an entity, representative, proxy or object. The handler is a routine for handling a general or relatively simple situation or operation, for example error elimination or data movements. In specific object-oriented programming languages that support messages, a handler represents a sub-program that processes a message for a specific object class.

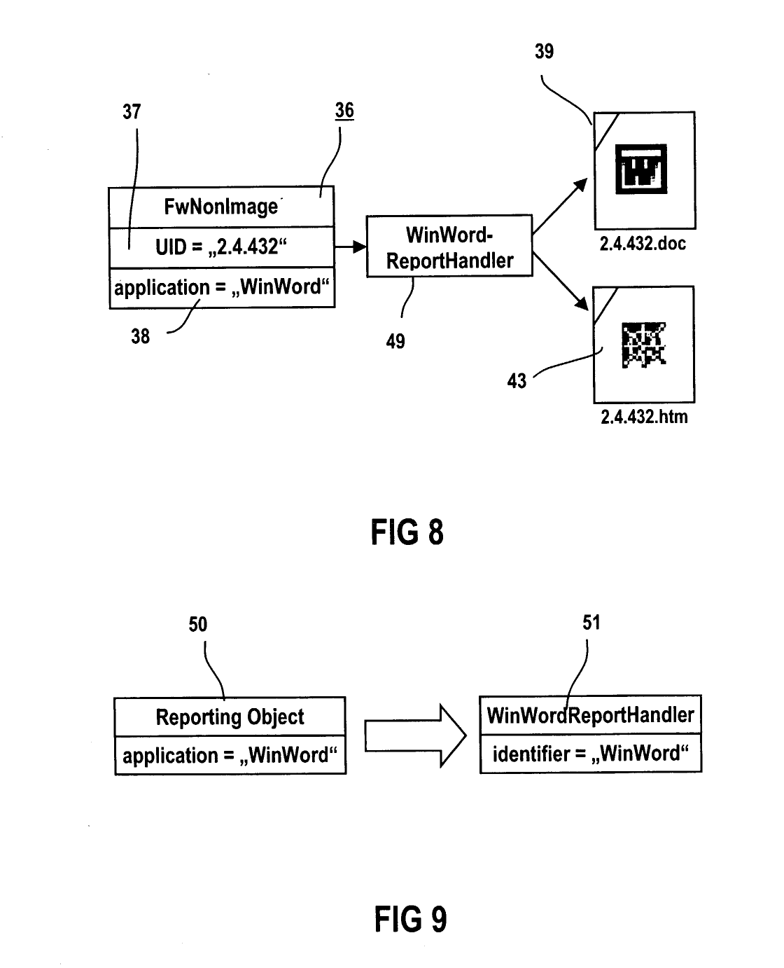

In FIG. 8, a WinWord report handler 49 is arranged between the first generic FNI object 36 shown in FIG. 6 and the FwReport 39 or, respectively, the WinWord HTML representation 44.

Each report handler has a system-wide, unambiguous string identifier that represents the application triggered by the report handler. This identifier must define the application of the report object that is controlled by the report handler.

As indicated in FIG. 9, however, this means that a report object 50 of the WinWord application merges into a WinWord report handler 51 with an identifier WinWord.

Each report handler must have itself registered at the handler dispatcher, a single-element set, that process the report handler by means of its identifier. The handler dispatcher is a central service that allocates reporting applications to reports. This software component knows all available reporting applications and allocates these to the appertaining reporting application on the basis of features of the reports.

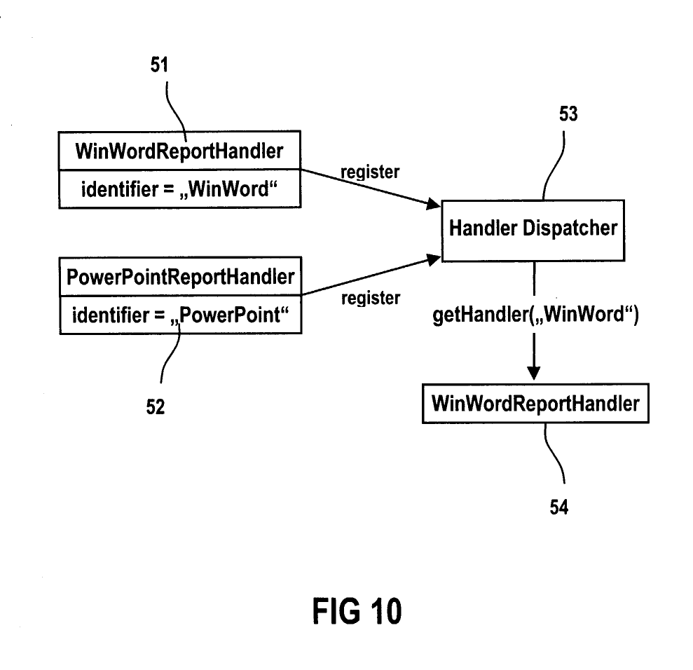

This registration is shown in FIG. 10, wherein the WinWord report handler 51 having the identifier WinWord and a PowerPoint report handler 52 having the identifier PowerPoint have themselves registered at a handler dispatcher 53. The handler dispatcher 53 then assigns the appertaining report handler, the WinWord report handler 54 in this case. The report applications are thereby “wrapped”, i.e. represented as a proxy, by what are referred to as report handler objects.

The following, exemplary scenario can occur when editing a report:

1.) First, the application properties of the report object 24 are read.

2.) An inquiry for a handler 54 with the same identification as the application properties is then made at the handler dispatcher 53.

3.) The unambiguous identifier 25 of the report object 24 is read out.

4.) Last, the editing method of the handler is called with the unambiguous identifier 25 as argument.

The inventive fashioning of the medical system is based on the encapsulation of all components for generating findings by means of generic interfaces, so that a close integration is enabled on the one hand but, on the other hand, the medical image processing system can operate exclusively on generic interfaces and, thus, a maximum flexibility is assured.

The invention has the following embodiments:

Generic object for link-in and generation of a DICOM SR (structure reporting) using a DICOM standard for imaging clinical reports.

Generic link-in of findings in WinWord

Generic link-in of knowledge-base-controlled diagnosis tools

Generic link-in of HIS databanks

Appendix of abbreviations employed herein:

DICOM Digital Imaging and Communications in Medicine DICOM standard is an industrial standard for the transmission of images and further medical information between computers, so that a digital communication is possible between diagnosis and therapy devices of different manufacturers.

DICOM SR DICOM Structured Reporting DICOM standard for imaging clinical reports.

EPR Electronic patient record

FNI FwNonImage, Framework NonImage An object of a framework that does not represent an image

ID Identification Numbers, names or other unambiguous features for recognizing specific devices, users, persons, general procedures or other elements in a computer or in a program

HIS Hospital Information System System for general hospital management, having the principal features of patient management, bookkeeping and accounting, personnel management, etc.

PACS Picture Archival and Communication System

RIS Radiology Information System: Information system for data management with the radiology department that, for example, supports the patient access, the creation of work lists, the reporting, report management, bookkeeping and accounting, etc.

UID Send-ID (Unique Identifier)

void A method that returns no result

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical system architecture comprising:
a workstation comprising an image processing system based on a component-oriented architecture; and
means for producing and manipulating text information linked into said imaging processing system by generic objects and interfaces allowing said means for producing and manipulating text information to be flexibly replaced without modification of said image processing system.

2. A medical system architecture as claimed in claim 1 wherein said workstation acquires information selected from the group consisting of medical data and medical examination images.

3. A medical system architecture as claimed in claim 1 wherein said workstation post-processes information selected from the group consisting of medical data and medical examination images.

4. A medical system architecture as claimed in claim 1 wherein said text information is represented by one of said generic objects containing unique features for identification of said text and identification of said means for producing and manipulating text information.

5. A medical system architecture as claimed in claim 1 wherein said image processing system identifies said means for producing and manipulating text information and controls said means for producing and manipulating text information using a component, specific to said means for producing and manipulating text information, that is linked to said image processing system.

6. A medical system architecture as claimed in claim 1 wherein said means for producing and manipulating text information are represented by a generic component, as one of said generic objects, with generic methods, functions and interfaces.

7. A medical system architecture as claimed in claim 1 wherein said means for producing and manipulating text information are represented by a generic component with generic methods, as one of said generic objects.

8. A medical system architecture as claimed in claim 1 wherein said means for producing and manipulating text information comprises a text processing system.

9. A medical system architecture as claimed in claim 1 wherein said means for producing and manipulating text information are selected from the group consisting of tools, applications and systems.

10. A medical system architecture as claimed in claim 1 wherein said means for producing and manipulating text information comprise applications.

11. A medical system architecture comprising:
a workstation comprising an image processing system based on a component-oriented architecture;
interchangeable means for producing and processing text information having an object allocated thereto containing identification criteria and references for the means for producing and processing text information allocated thereto;
a handler dispatcher for identification of one of said means for producing and processing text information based on said identification criteria; and
a control device for said means for producing and processing text information based on said references using a component linked to said image-processing system that is specific for the means for producing and processing text information identified by said identification criterion.

12. A medical system architecture as claimed in claim 11 wherein said workstation acquires information selected from the group consisting of medical data and medical examination images.

13. A medical system architecture as claimed in claim 11 wherein said workstation post-processes information selected from the group consisting of medical data and medical examination images.
